(19) United States
(12) Patent Application Publication
(10) Pub. No.: US 2026/0000689 A1
(43) Pub. Date: Jan. 1, 2026

(54) TARGETING CREB-HEPARAN SULFATE-FGF PATHWAY FOR CANCER TREATMENT

(71) Applicant: UNIVERSITY OF SOUTH FLORIDA, Tampa, FL (US)

(72) Inventors: Hua YANG, Tampa, FL (US); Lianchun WANG, Tampa, FL (US)

(21) Appl. No.: 18/881,110

(22) PCT Filed: Jul. 6, 2023
(86) PCT No.: PCT/US2023/069689

§ 371 Date: Jan. 3, 2025

Related U.S. Application Data
(60) Provisional application No. 63/367,782, filed on Jul. 6, 2022.

(51) Int. Cl. A61K 31/661; A61K 31/167; A61K 31/337; A61K 31/4155; A61K 31/4439; A61K 31/4709; A61K 31/497; A61K 31/58; A61K 38/46; A61P 35/00; C12N 15/113

(52) U.S. Cl. CPC A61K 31/661; A61K 31/167; A61K 31/337; A61K 31/4155; A61K 31/4439; A61K 31/4709; A61K 31/497; A61K 31/58; A61K 38/46; A61P 35/00; C12N 15/1137; C12Y 204/01225; C12N 2310/531

(57) ABSTRACT

The present disclosure provides for a method of treating a Pten-loss induced cancer in a subject in need thereof, the method comprising administering an inhibitor of a CREB-heparan sulfate-FGF axis.

### CROSS-REFERENCE TO RELATED APPLICATIONS

This application claims the benefit of priority to, and the benefit of, U.S. Provisional Application No. 63/367,782 filed on Jul. 6, 2022, the disclosure of which is hereby expressly incorporated by reference herein in its entirety.

### STATEMENT OF GOVERNMENT SUPPORT

This invention was made with government support under Grant No. U01CA225784, awarded by the National Cancer Institute, and Grant No. CA225784, awarded by the National Institutes of Health. The government has certain rights in the invention.

### BACKGROUND

Prostate cancer (PCa) is one of the most prevalent forms of malignancy and the second most common cause of cancer-related death in men. Prostate cancer is a type of cancer that occurs in the prostate, which is a small walnut-shaped gland in males that produces the seminal fluid that nourishes and transports sperm. There are varying types of prostate cancer, some of which grow slowly and are confined to the prostate gland, while there are other types that are aggressive and can spread quickly. Prostate cancer can also metastasize to nearby organs such as the bladder, or travel through the bloodstream or lymphatic system to bones or other organs. Historically, the factors that drive the expression of PCa related genes and the functional consequence of those genes in PCa pathogenesis has remained unclear. There is a need for a better understanding of PCa pathogenesis so that it can be utilized to improve efforts to cure this life-threatening disease.

The methods disclosed herein address these and other needs.

### SUMMARY

In accordance with the purposes of the disclosed materials and methods, as embodied and broadly described herein, the disclosed subject matter, in one aspect, cancer treatment.

Thus, in one example, a method of treating a Pten-loss induced cancer in a subject in need thereof is provided, including administering an inhibitor of a CREB-heparan sulfate-FGF axis.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIG. 1A-FIG. 1D show an aberrant heparan sulfate (HS) expression occurs in prostate cancer and is associated with loss-of-function Pten mutation (FIG. 1A, FIG. 1B). The comparison of HS gene expression in normal prostate epithelial cells (PECs) vs PCa PECs (FIG. 1A) and benign PECs via PCa PECs (FIG. 1B) in human PCa specimens. The data were extracted from Tomlins' microarray of laser-capture microdissection isolated PECs, including 101 specific cell populations from 44 individuals. Tomlins, 2007. FIG. 1C shows bioinformatics analysis using Enrichr found Pten, the tumor suppressor which loses is function is commonly seen in PCa patients, is associated with both PCa and Proteoglycans in cancer. FIG. 1D shows the Cancer Genome Atlas (TCGA) data analysis observed several HS and HS proteoglycan gene expression is upregulated in the Pten abnormal PCa patients. Analysis of RNAseq data from 248 Pten normal and 76 Pten abnormal (down, mut, homedel) patients.

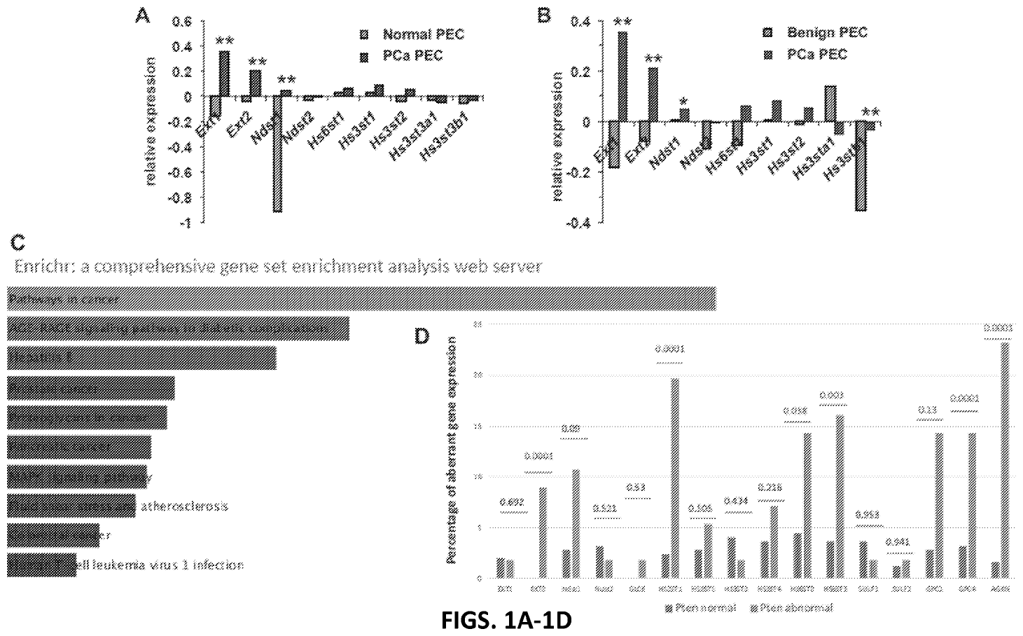

FIG. 2A-FIG. 2B show Pten loss-of-function upregulates HS expression in PCa in mice and in cultured human prostate epithelial cells. FIG. 2A shows prostate-specific Pten knockout mice, a spontaneous PCa mouse model, recapitulate the aberrant HS expression in human PCa. PCa pathological processes in ΔPten mice (up panel) and qRT-PCR analysis of HS biosynthesis gene expression in ΔPten and wildtype littermate prostates at ages of 6 weeks. The data were presented after normalization to the wildtype control. N=3 mice per group. *, P<0.05; **, P<0.005. FIG. 2B shows that Pten knockdown upregulates Ext1 and Ndst1 in human PEC line pNT2. Similar data are seen in primary human PECs and HEK293 cells.

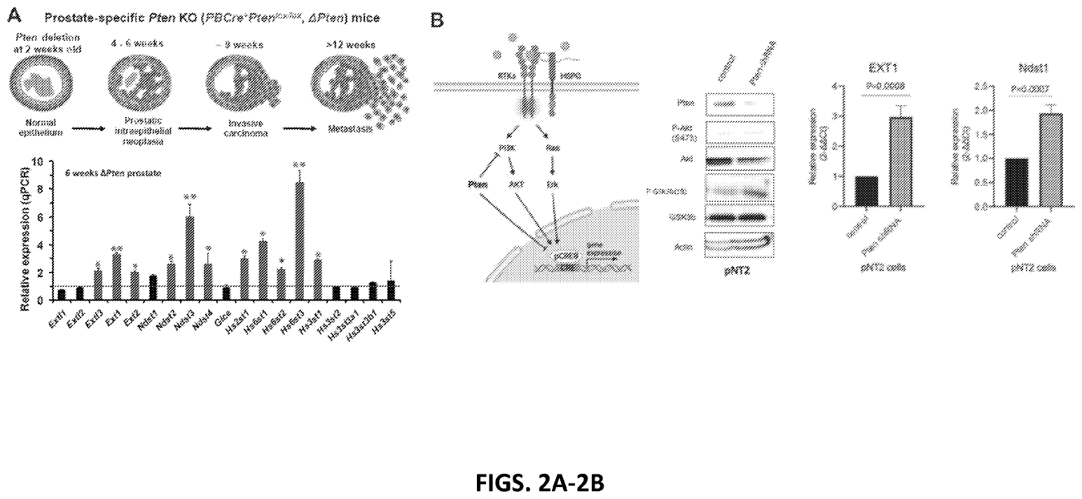

FIG. 3A-FIG. 3C show Pten-loss upregulated HS gene expression is mediated by activating CREB pathway, not AKT signaling in pNT2 cells. FIG. 3A shows treatment of pNT2 cells with TCN, blocked AKT phosphorylation but did not affect Pten-loss upregulated HS gene expression. FIG. 3B shows knockdown of transcription factor CREB blocked Pten-loss upregulated HS gene expression as well as the known CREB target gene NR4A2. FIG. 3C shows the activation of CREB promoter using the CRISPR/Ca9 Synergistic Activation Mediator (SAM) system upregulated both NR4A2 and Ext1 expression.

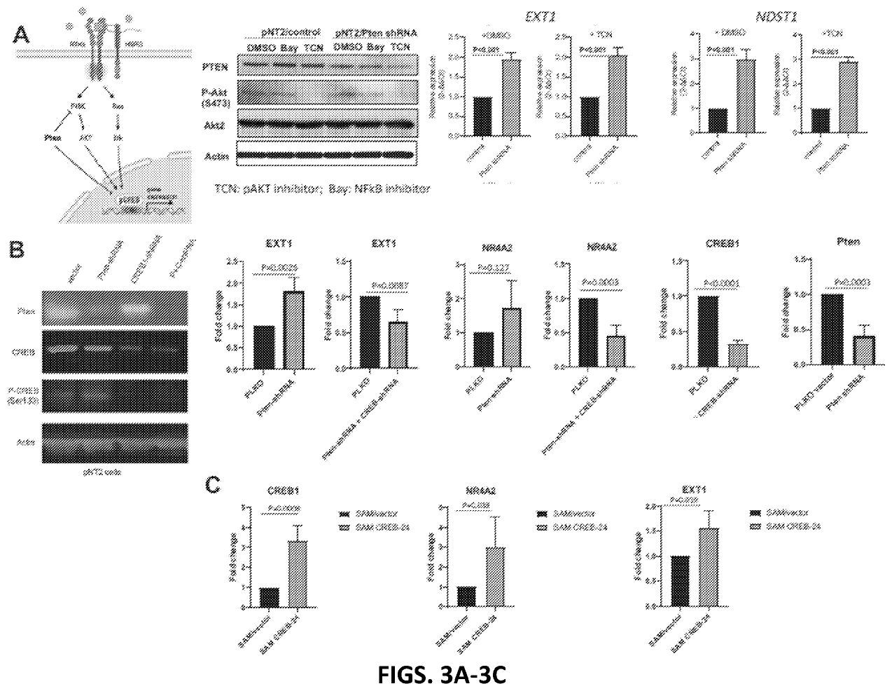

FIG. 4A-FIG. 4X show HS is required for PCa tumorigenesis. FIG. 4A-FIG. 4D show Ext1 and HS expressions in prostates. Cryosections of 5-week-old prostates were co-stained with anti-Ext1 antibody (FIG. 1A, FIG. 1B) or anti-HS antibody (FIG. 1C, FIG. 1D) and DAPI. FIG. 4E-FIG. 4J show anatomical (5-week-old, FIG. 4E, FIG. 4F), macroscopic (branching morphogenesis, 5-week-old; FIG. 4G, FIG. 4H) and histological (10-week old; FIG. 4I, FIG. 4J) analyses of prostates. FIG. 4K to FIG. 4V show normal structures and invasive cells are pointed to by black and yellow arrowheads, respectively. At 13 weeks of age, the ΔExt1ΔPten prostates remain their epithelial morphology, wherein the ΔPten prostate cells change to mesenchymal cell morphology. FIG. 4W-FIG. 4X show the anterior prostates and their weight. N=4-5 mice per group. AP, anterior prostate; DLP, dorsolateral prostate; VP, ventral prostate.

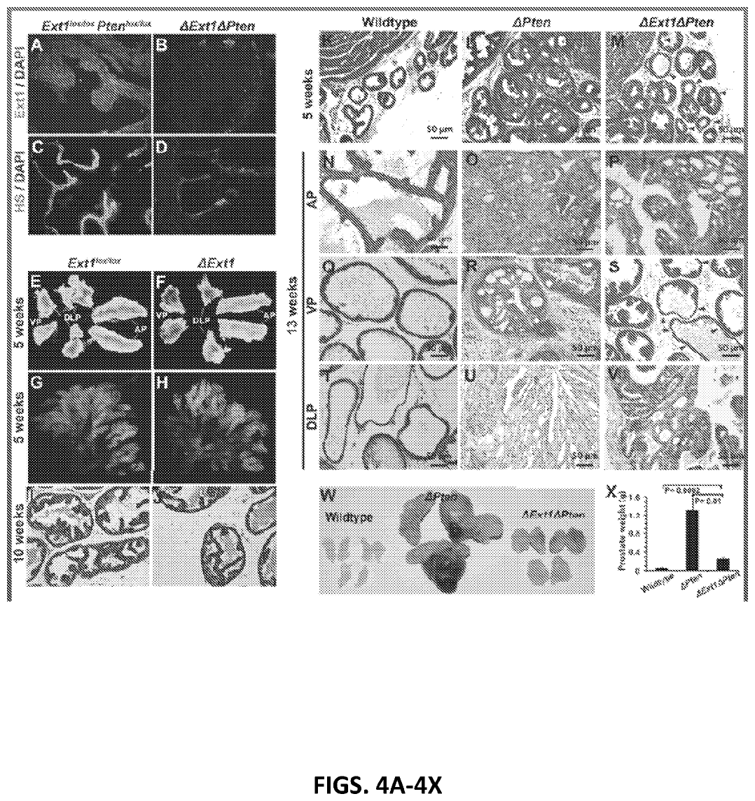

FIG. 5A-FIG. 5C to show HS is required for prostate cancer cell growth in vitro and in vivo in a xenograft mouse model; overexpression of EXT1 increased prostate cancer cell growth in vitro, but the effect is not obvious when the cells were injected at a high cell number. FIG. 5A shows knockdown of EXT1 attenuated prostate cancer cell line Myc-CAP growth in culture, whereas EXT1 overexpression increased the cell growth. FIG. 5B shows knockdown of EXT1 attenuates Myc-CAP cell growth in the immune-competent syngeneic FVB/NJ mice. Myc-CAP cells (1×10⁶) were s.c. injected per site. FIG. 5C shows overexpression of EXT1 did not obviously increase Myc-CAP cell growth in the immune-competent syngeneic FVB/NJ mice when the cells were s.c. injected at the dose of 1×10⁶ cells per site.

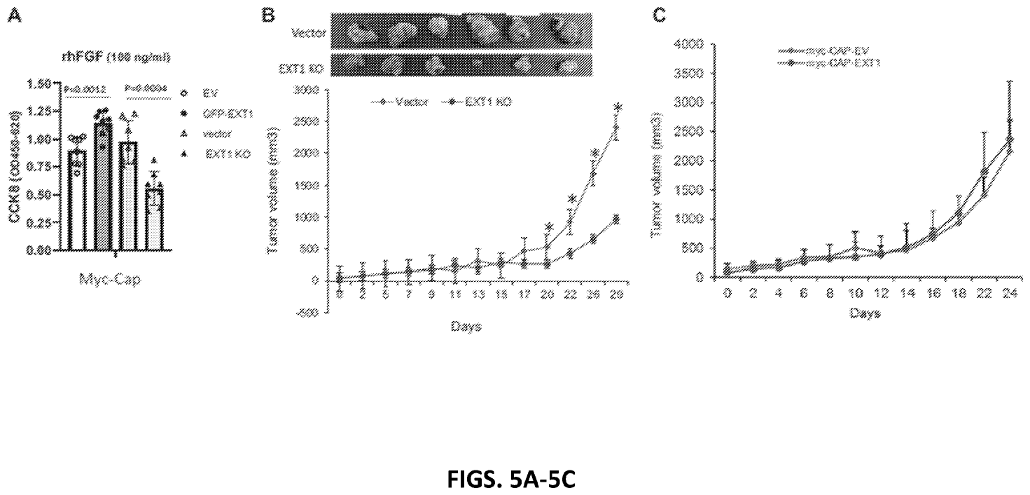

FIG. 6A-FIG. 6G show EXT1 overexpression induces prostate epithelial cell colony formation in soft agar-promotes prostate cancer initiation in vitro. The RWPE-1 cells, an immortalized human prostate epithelial cell line, were transduced with adenovirus containing empty vector (EV) (FIG. 6A), EXT1 (FIG. 6B), or myristoylated AKT1 (myr-AKT) (FIG. 6C), and then cultured in soft agar for 19 days. myr-AKT was included as a positive control. FIG. 6D shows a graph of the number of colonies for each of EV, EXT1, and myr-AKT. FIG. 6E shows a 4-times enlarged picture of the human prostate epithelial cell line of FIG. 6B. FIG. 6F-FIG. 6G show 20-times enlarged pictures of the human prostate epithelial cell line of FIG. 6B.

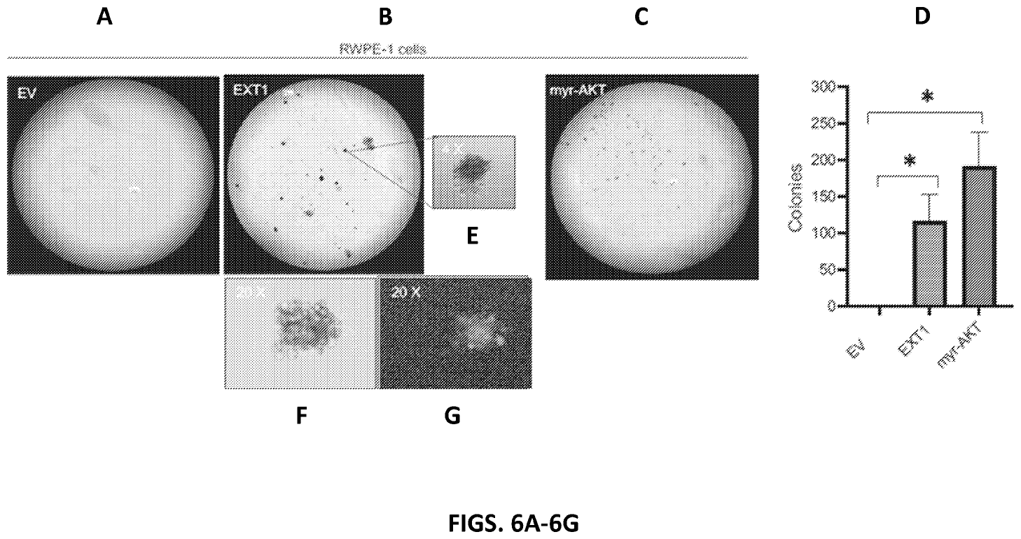

FIG. 7A-FIG. 7F show inhibition of CREB or HS attenuates prostate cancer growth. FIG. 7A-FIG. 7D show the CREB inhibitor KG-501 does-dependently inhibited the expression of EXT1 mRNA 9FIG. 7A) and HS (FIG. 7B) in Myc-CaP cells, and the in vitro cell growth (FIG. 7C) and migration (FIG. 7D). FIG. 7E-FIG. 7F show KG-501 and Surfen, an HS inhibitor, efficiently inhibited the growth of Myc-CaP cells without (FIG. 7E) or with EXT1-overexpression (FIG. 7F) in an s.c. xenograft prostate cancer mouse model.

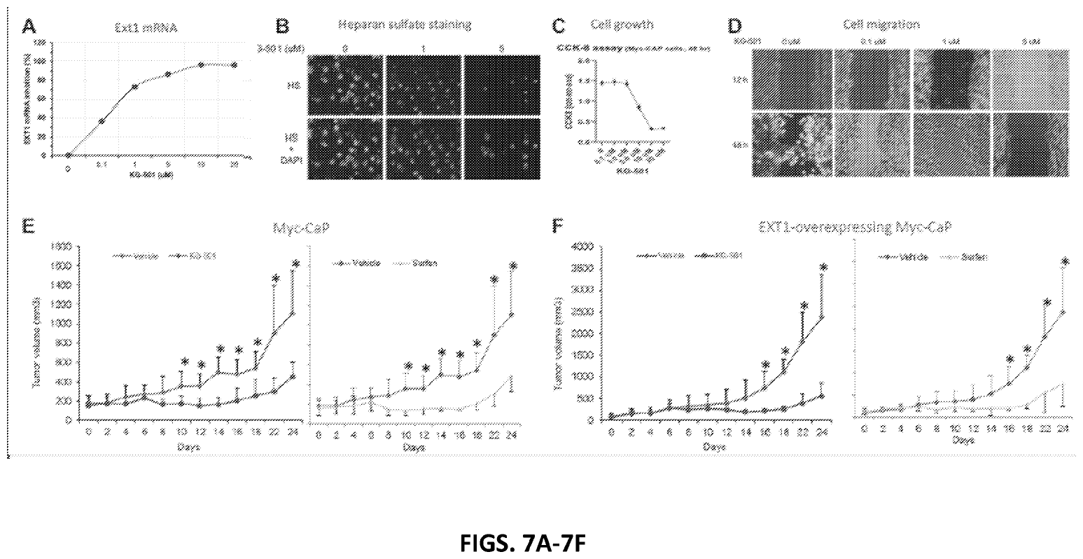

FIG. 8 shows a heparan sulfate proteoglycan (HSPG).

FIG. 9 shows a linear polysaccharide with multiple modifications which generate ligan-binding sites (fine structures).

FIG. 10 shows aberrant HS gene expression in breast, prostate, and colon cancers. N=16-24 for each tumor type. Tumor tissue was greater than 70%, with en bloc tissue for RNA isolation. Analyzes was performed via multiplex RT-PCR. Shuovskih, 2015.

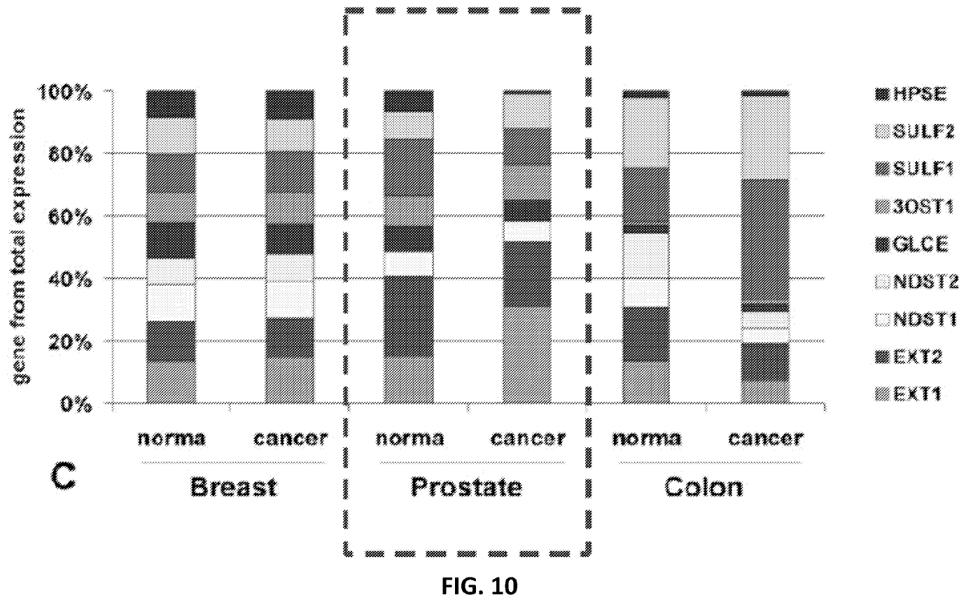

FIG. 11A-FIG. 11B show microarrays of laser-capture microdissection isolated prostate epithelial cells (PEC). The comparison of normal PECs vs PCa PECs (FIG. 11A) or benign PECs vs PCa PECs (FIG. 11B) in human PCa specimens. The study included 101 specific cell populations from 44 individuals. Tomlins, 2007.

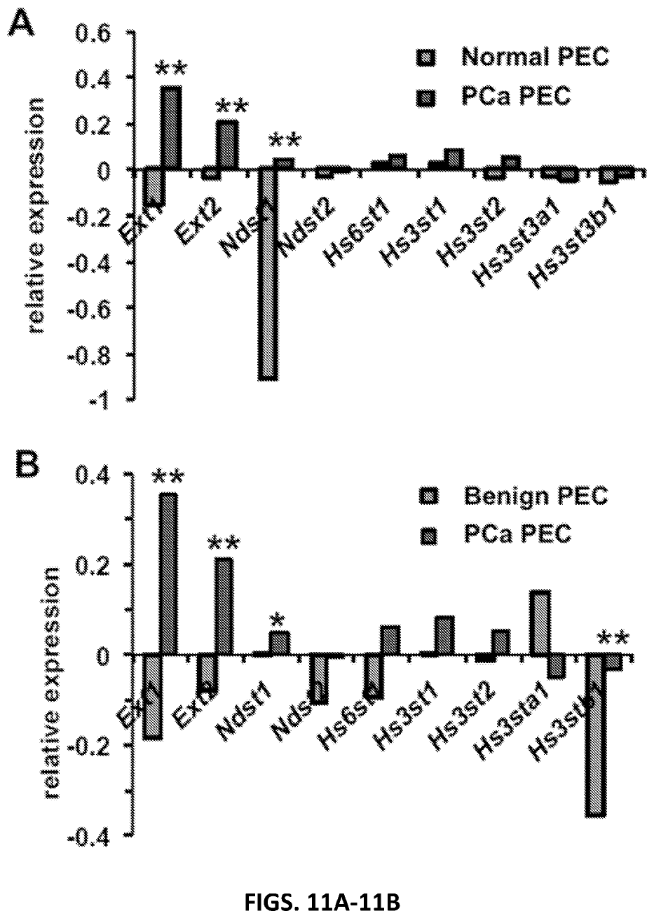

FIG. 12A-FIG. 12B show prostate-specific Pten knockout mice, a spontaneous PCa mouse model, recapitulate the aberrant HS expression in human PCa. FIG. 12A shows a prostate-specific Pten Knockout (PBCre+Ptenlox/lox, ΔPten) mice cell. FIG. 12B shows the relative expression of select genes at 6 weeks ΔPten prostate.

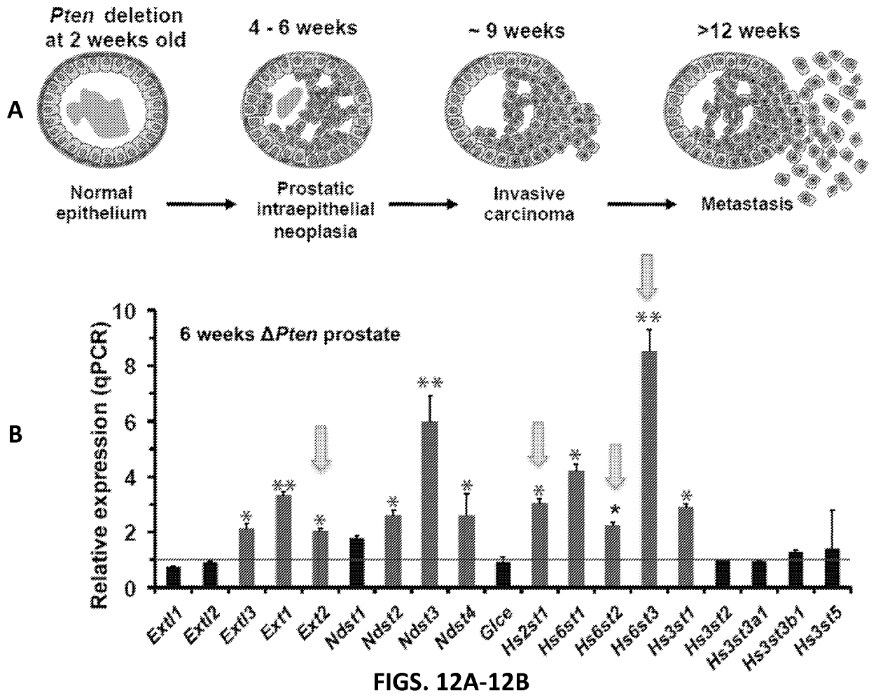

FIG. 13A-FIG. 13B show that Pten-loss upregulates HS gene expression in human prostate epithelial cells. FIG. 13A shows primary human prostate epithelial cells, FIG. 13B shows a human prostate epithelial cell line.

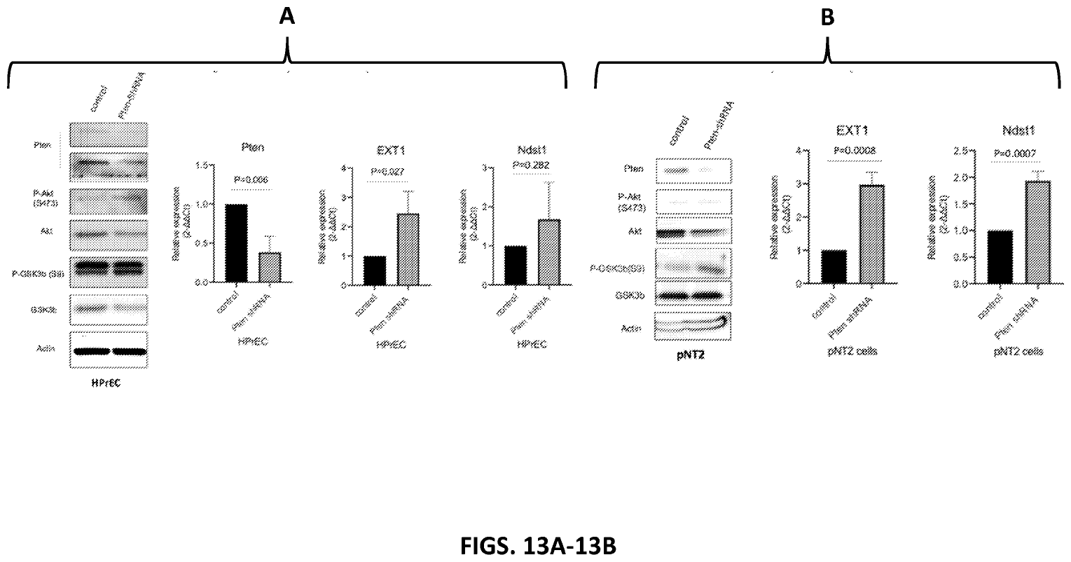

FIG. 14A-FIG. 14C show that inhibition of AKT did not alter Pten-loss-induced HS gene expression. FIG. 14A shows pharmacological treatment with pNT2 cells (normal human prostate epithelial cells, do not form colony in vitro or grow in xenograft transplantation in vivo). FIG. 14B shows the relative expression of EXT1 with dimethylsulfoxide (DMSO) and triciribine (TCN). FIG. 14C shows the relative expression of NDST1 with DMSO and TCN.

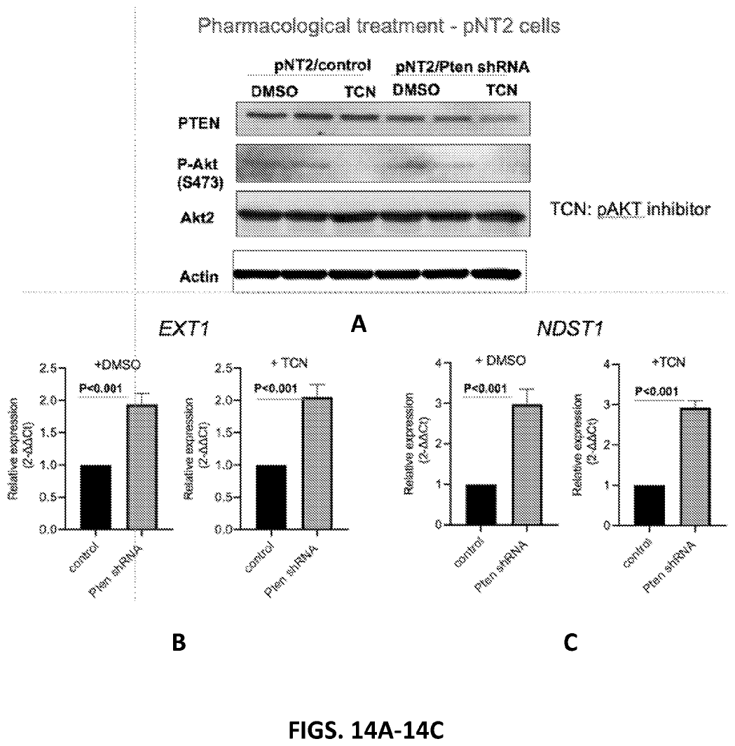

FIG. 15A-FIG. 15F show knockdown of CREB blocks Pten-loss induced HS gene expression and CREB knockdown in pNT2 cells. FIG. 15A and FIG. 15B show EXT1 fold change in pNT2 cells. FIG. 15C and FIG. 15D show NR4A2 fold change in pNT2 cells. FIG. 15E shows CREB1 fold change in pNT2 cells. FIG. 15F shows Pten fold change in pNT2 cells.

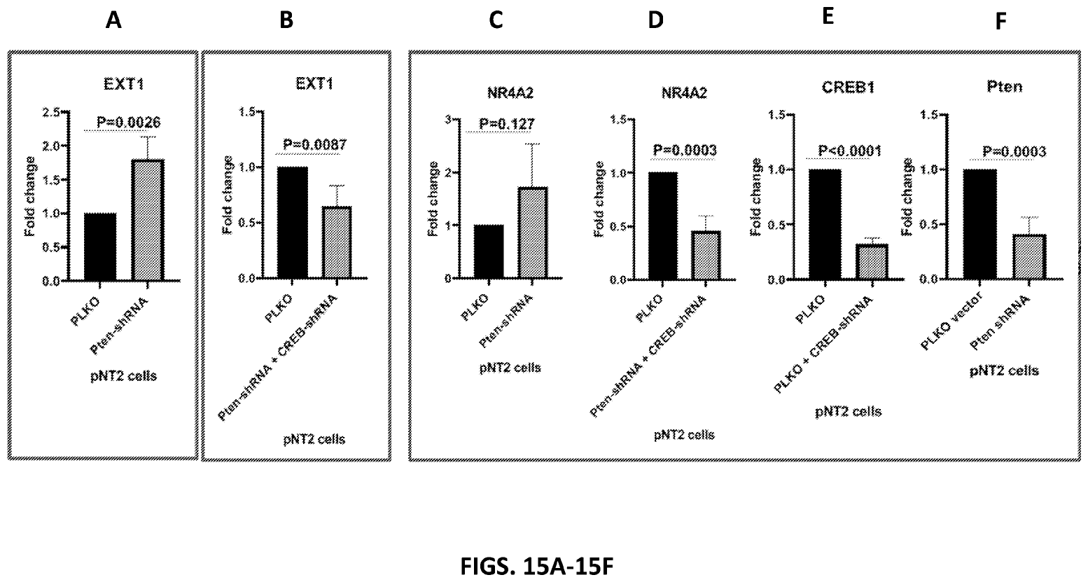

FIG. 16A-FIG. 16C show Pten-loss induced HS gene expression alteration was mediated by CREB pathway, using CRISPR/Cas9 Synergistic Activation mediator (SAM) system. FIG. 16A shows CREB1 gene fold change in pNT2 cells. FIG. 15B shows NR4A2 gene fold change in pNT2 cells. FIG. 15C shows EXT1 fold change in pNT2 cells.

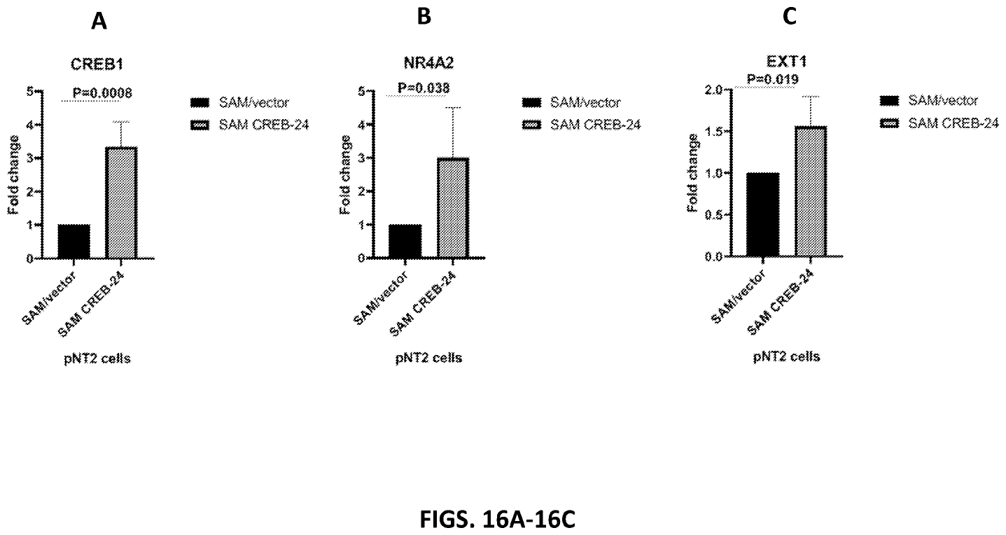

FIG. 17A-FIG. 17B show colony formation in soft agar. FIG. 17A shows immortalized human prostatic epithelial cell line (RWPE-1) which can be induced to be tumorigenic and used for tumor initiation study. FIG. 17B shows RWPE-1 and RWPE-1/THP-1 colony/yield. Yang, 2013.

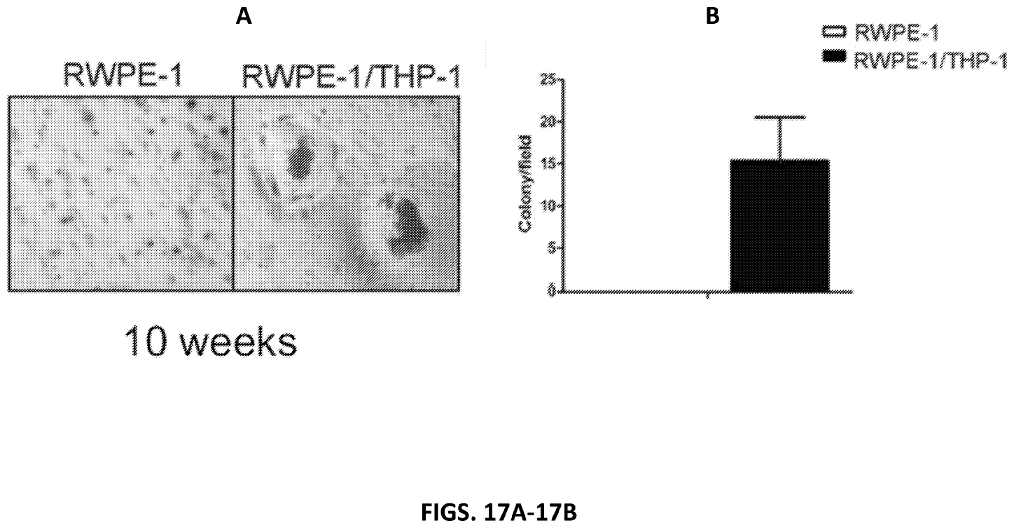

FIG. 18A-FIG. 18D show a xenograft model. FIG. 18A shows mice with RWPE-1 induced to be tumorigenic. FIG. 18B shows the tumors removed from the mice. FIG. 18C shows a corresponding hematoxylin and eosin stain (H&E stain). FIG. 18D shows the tumor count for the respective mice.

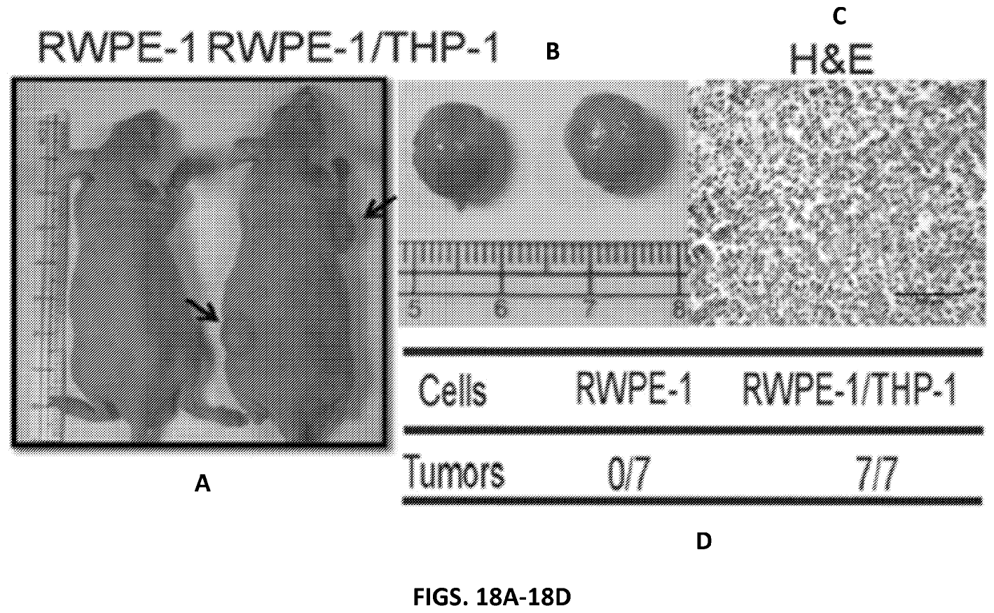

FIG. 19A-FIG. 19C show that EXT1 overexpression induced prostate epithelial cell RWPE1 colony formation in soft agar. FIG. 19A shows RWPE-1 and EV, FIG. 19B shows RWPE1 and EXT1, and FIG. 19C shows RWPE1 and EXT1 with heparanase (HPSE) treatment.

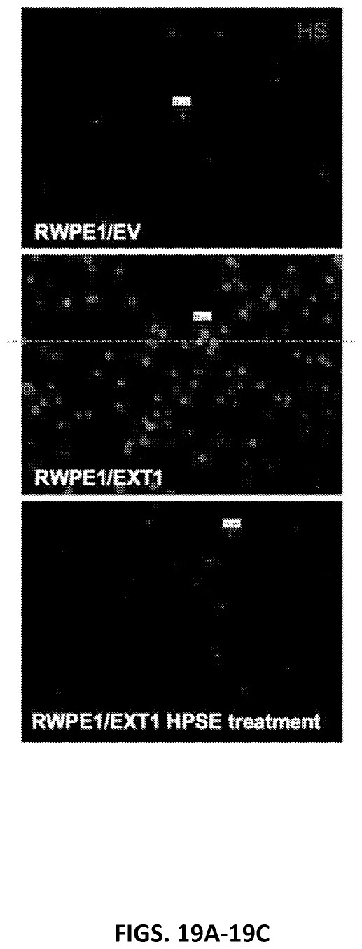

FIG. 20A-FIG. 20D show EXT1 overexpression initiated rWPE1 cells to form tumors in vivo (nude mice). FIG. 20A show the number of tumors for specific types of cells with 1×10⁷ cells per injection over 36 days. FIG. 20B shows the nude mice with RWPE1 cells and EV. FIG. 20C shows the nude mice with RWPE1 cells and EXT1, with the corresponding tumors and H&E stain. FIG. 20D shows RWPE1 cells and myr-Akt, with the corresponding tumors and H&E stain.

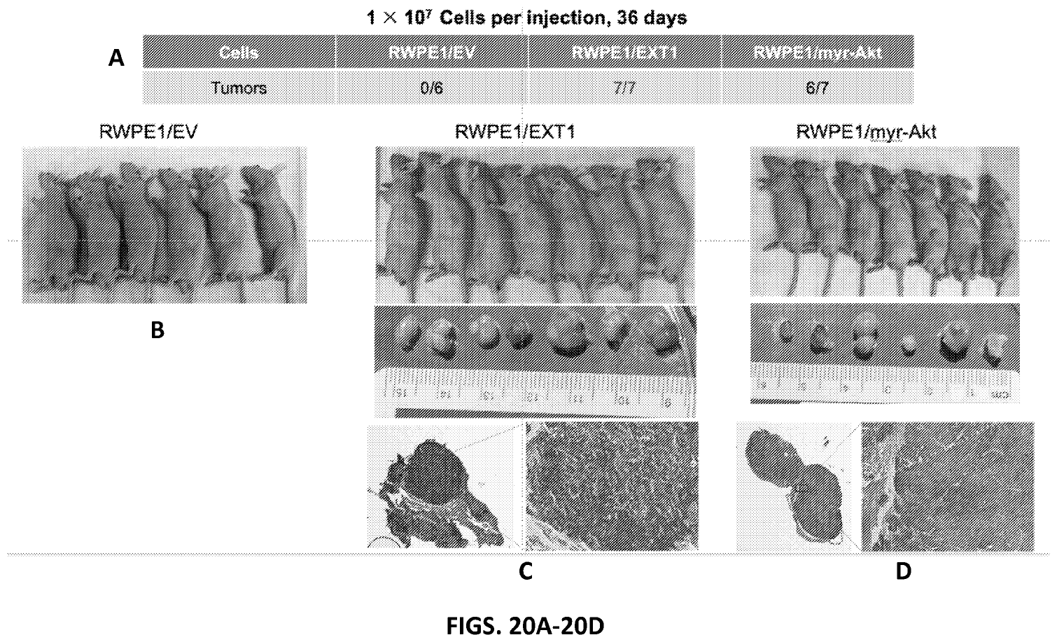

FIG. 21A-FIG. 21C show that EXT1 overexpression promoted prostate cancer growth in the Myc-CaP xenograft model, wherein Myc-CaP cell line is derived from spontaneous prostate cancer in c-Myc transgenic mice, androgen dependent, and tumorigenic in immune competent syngeneic FVB/NJ mice. FIG. 21A shows tumor volume over time with EXT1 knockout. FIG. 21B shows tumor volume over time with 1×10⁶ cells per injection with myc-CAP-EXT1 and myc-CAP-EV. FIG. 21C shows tumor volume over time with 1×10⁵ cells per injection for myc-CAP-EXT1 and myc-CAP-EV.

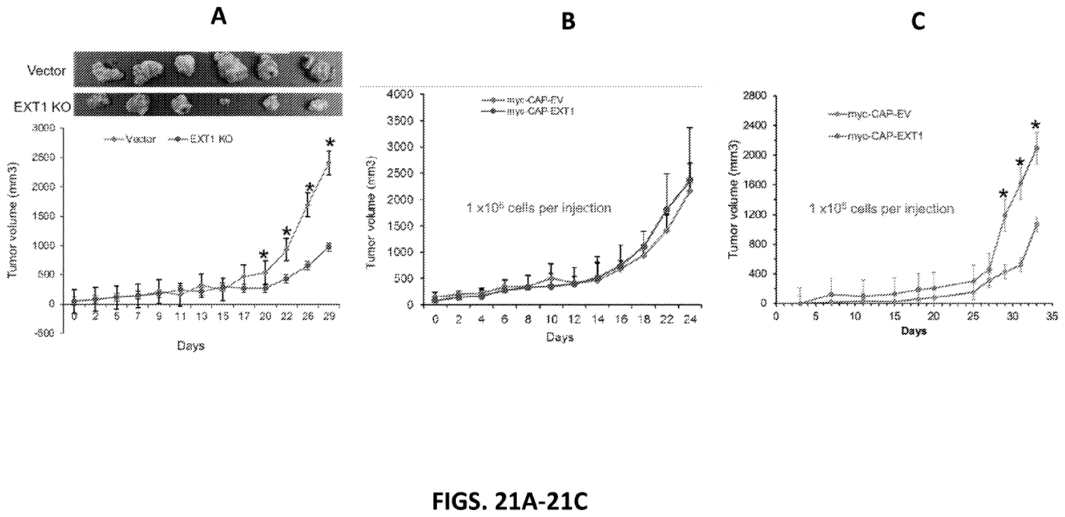

FIG. 22A-FIG. 22H show that inhibition of CREB and HS attenuated prostate cancer growth in vivo. FIG. 22A shows Ext1 mRNA inhibition. FIG. 22B shows heparan sulfate staining. FIG. 22C shows cell growth. FIG. 22D shows cell migration. FIG. 22E and FIG. 22F show myc-Cap cells in vivo for KG-501 (FIG. 22E) and Surfen (FIG. 22F). FIG. 22G and FIG. 22H show Ext1-overexpressing myc-Cap cells in vivo for KG-501 (FIG. 22G) and Surfen (FIG. 22H).

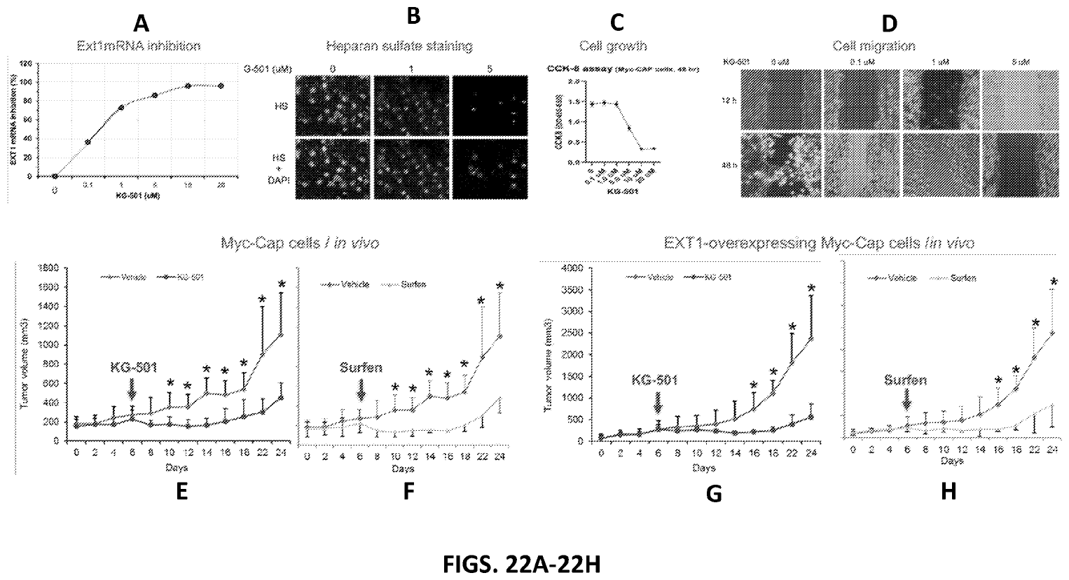

FIG. 23A-FIG. 23B show the molecular pathways that HS modulates to mediate Pten-loss-induced prostate tumorigenesis. FIG. 23A shows FGF/FGFR system in the prostate. Giacomini, 2021. FIG. 23B shows in vitro cell growth.

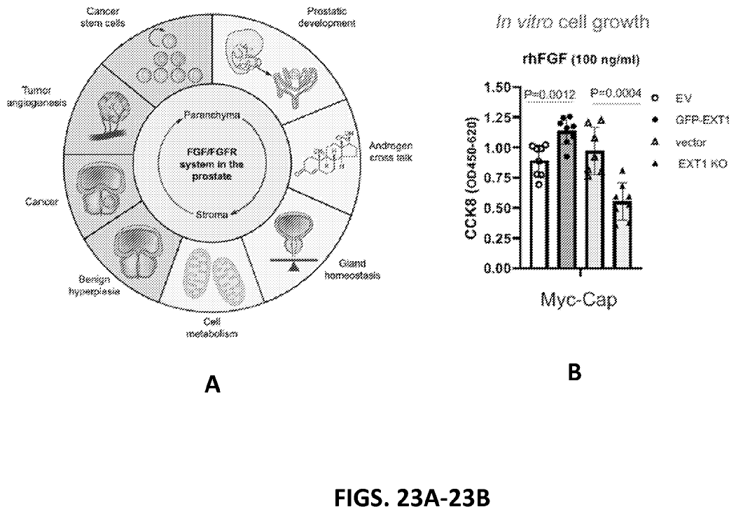

FIG. 24A-FIG. 24D show that EXT1-overexpressing MyC-CaP cells are more resistant to FGFR inhibitor in colony formation. FIG. 24A shows EXT1 and EV with DMSO. FIG. 24B shows EXT1 and EV with 1 μm AZD4547. FIG. 24C shows EXT1 and EV with 5 μm AZD4547. FIG. 24D shows EXT1 and EV with 10 μm AZD4547.

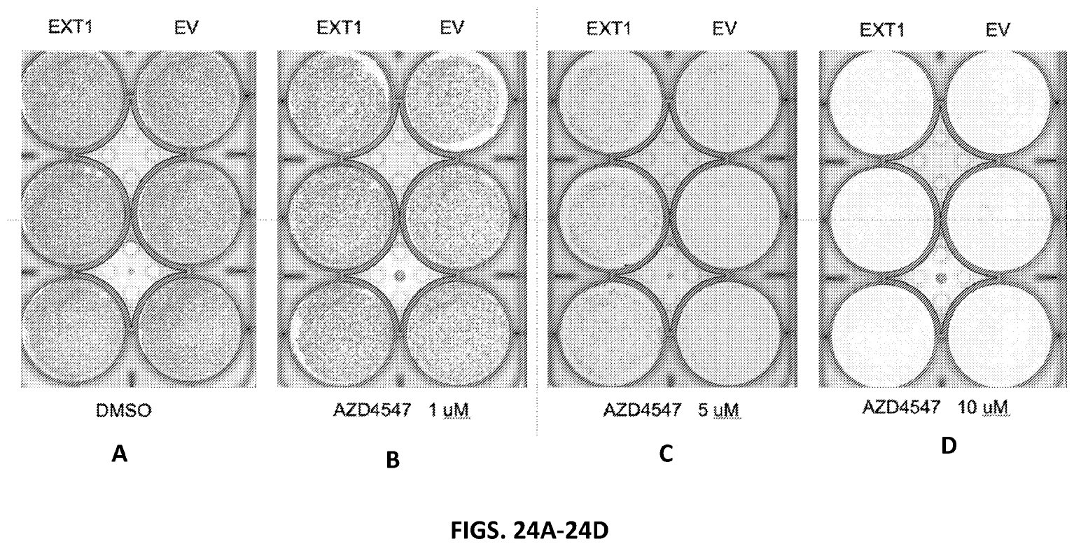

FIG. 25A-FIG. 25B show that AZD4547 blocks EXT1-overexpression-enhanced prostate cancer growth in vivo. FIG. 25A shows tumor volume over time for EV-V and EV-AZD. FIG. 25B shows tumor volume over time for EXT1-V and EXT1-AZD.

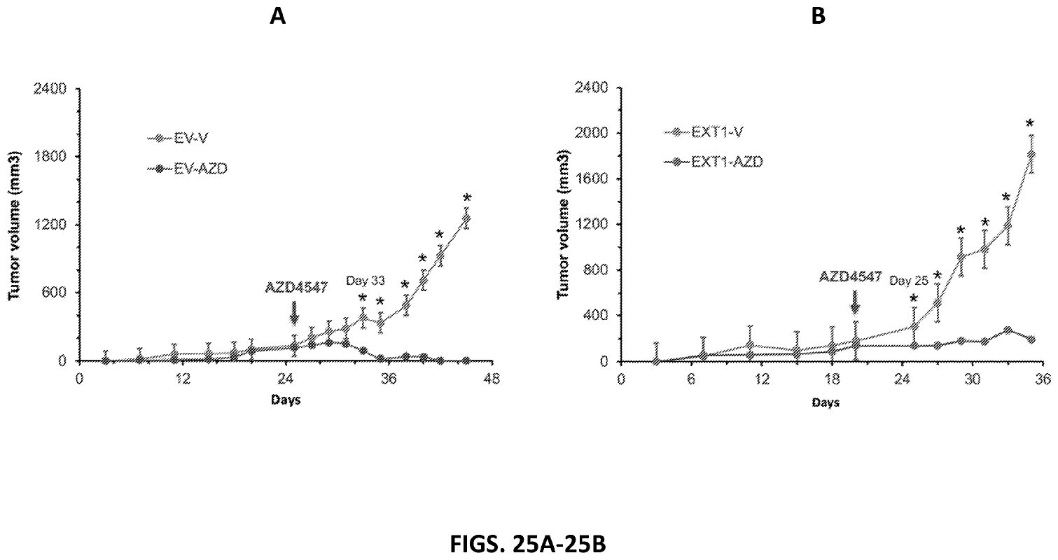

FIG. 26 shows Pten-loss activates CREB-HS_FGF signaling pathway to initiate and promote prostate tumorigenesis. Upregulated HS boosted oncogenic AKT and ERK signaling.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

The term “ion.” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl. 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure-CH═CH₂; 1-propenyl refers to a group with the structure-CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-penoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z′C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

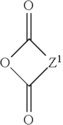

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻.

A “carbonate ester” group as used herein is represented by the formula Z¹OC(O)OZ².

The term “cyano” as used herein is represented by the formula —CN.

The term “ester” as used herein is represented by the formula —OC(O)Z¹ or C(O)OZ¹, where Z¹ can be an alkyl, alkenyl, alkynyl, aryl, heteroaryl, cycloalkyl, -cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

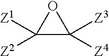

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a.” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

The term “subject” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

The term “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. Further, if a treatment can treat a disease in a subject having symptoms of the disease, it can also prevent or suppress that disease in a subject who has yet to suffer some or all of the symptoms.

The term “therapeutically effective” refers to the amount of the composition used is of sufficient quantity to ameliorate one or more causes or symptoms of a disease or disorder. Such amelioration only requires a reduction or alteration, not necessarily elimination.

### Method

### Method of Treating Pten-Loss Induced Cancer

The present disclosure, in one aspect, provides for a method of treating a Pten-loss induced cancer in a subject in need thereof, the method comprising administering an inhibitor of a CREB-heparan sulfate-FGF axis.

CREB is a transcription factor that plays a crucial role in regulating various cellular responses, including proliferation, survival, and differentiation. It is induced by a wide range of growth factors and inflammatory signals, and subsequently mediates the transcription of genes containing a cAMP-responsive element.

HS (heparan sulfate) is a linear polysaccharide that is highly sulfated. It is covalently linked to protein cores to form HS proteoglycans (HSPGs). HSPGs are found ubiquitously on cell surfaces, such as syndecans and glypicans, as well as in the extracellular matrix, including agrin, perlecan, and collagen XVIII. They interact with protein ligands through their HS chains, critically modulating cell-cell and cell-matrix interactions and signaling.

Fibroblast growth factors (FGFs) transmit signals through FGF receptors (FGFRs) and regulate a broad spectrum of biological functions, including cellular proliferation, survival, migration, and differentiation. HSPGs are known to act as co-receptors, facilitating FGF-FGFR signaling.

The loss of Pten activates CREB signaling, leading to increased HS expression on prostate cancer epithelial cells. This elevated HS expression enhances FGF signaling, thereby accelerating the pathogenesis of prostate cancer. Targeting the CREB-HS-FGF axis is an effective approach for treating prostate cancer and potentially other cancers driven by Pten-loss, CREB signaling, and/or FGF signaling.

In some examples, the inhibitor of the CREB-heparan sulfate-FGF axis comprises at least one inhibitor of CREB. CREB is a cellular transcription factor. It binds to CAMP response elements, thereby increasing or decreasing the transcription of genes. The following genes encode CREB or CREB-like proteins: CREB1, CREB2, CREB3, CREB5, CREB3L1, CREB3L2, CREB3L3, and CREB3L4.

In further examples, the inhibitor of CREB comprises KG501. KG-501 (2-naphthol-AS-E-phosphate) is a cAMP response element-binding protein (CREB) inhibitor that disrupts CREB-dependent transcription and CREB:CBP interaction. KG-501 has the chemical structure as shown in Formula I.

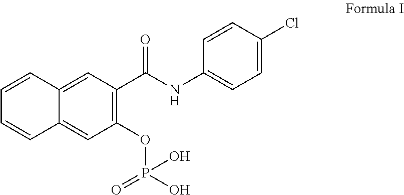 [SMILES: O=C(Nc1ccc(Cl)cc1)c1cc2ccccc2cc1OP(=O)(O)O]

In certain examples, the inhibitor of CREB comprises 666-15. 666-15 (3-(3-Aminopropoxy)-N-(2-((3-((4-chloro-2-hydroxyphenyl)carbamoyl)naphthalen-2-yl)oxy)ethyl)-2-naphthamide) is a potent and selective chemical inhibitor of CREB. 666-15 has the chemical structure as shown in Formula II.

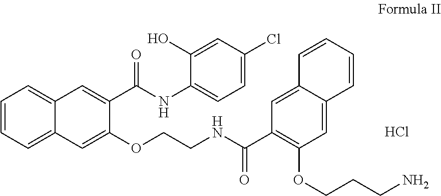 [SMILES: NCCCOc1cc2ccccc2cc1C(=O)NCCOc1cc2ccccc2cc1C(=O)Nc1ccc(Cl)cc1O ; Cl]

In specific examples, the inhibitor of the CREB-heparan sulfate-FGF axis comprises an inhibitor of heparan sulfate (HS) synthesis. The formation of HS takes place in the Gogli network, where most of the biosynthetic enzymes are anchored to the Golgi membrane. Biosynthetic precursors (3′-phosphoadenosine-5′-phosphosulfate [PAPS] and UDP-sugats_ are formed in the cytosol and transported into the Golgi. Prior to HS polymerization, the linkage region is formed attached to a serine residue in a core protein. Next, the EXT1/EXT2 polymerase complex adds alternating units of GlcNAc and GlcA to the non-reducing end of the growing chain (arrow a indicates the direction of polymerization). The polymerization is followed by a series of modification reactions, which can include N-deacetylation/N-sulfation, followed by epimerization and 2-O-sulfation, and finally 6-O- and 3-O-sulfation.

Genes that can impact HS synthesis can include, but are not limited to, EXT1, EXT2, Ndst1, Ndst2, Glce, Hs2st1, Hs3st1, Hs3st2, Hs3st4, Hs6st2, Hs6st3, Sulf1, Sulf2, or GPC2.

In some examples, the inhibitor of HS synthesis comprises bis-2-methyl-4-amino-quinolyl-6-carbamide. Bis-2-methyl-4-amino-quinolyl-6-carbamide, also referred to as Surfen, is a small molecule antagonist of heparan sulfate synthesis. Surfen has a chemical structure as shown in Formula III.

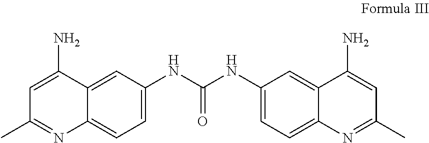

In further examples, the inhibitor of the CREB-heparan sulfate-FGF axis comprises an inhibitor of fibroblast growth factor (FGF). Fibroblast growth factors are a family of cell signaling proteins produced by macrophages; they are involved in a wide variety of processes, including development in animal cells. Irregularities in FGF function leads to a range of developmental defects. These growth factors can act as systemic or locally circulating molecules of extracellular origin that activate cell surface receptors. FGF can bind to heparin and to heparan sulfate. In humans, 23 members of the FGF family have been identified, all of which are structurally related signaling molecules. Some of the FGFs bind to fibroblast growth factor receptors (FGFRs), wherein the FGFR has three extracellular immunoglobulin-type domains (D1-D3), a single-span transmembrane domain and an intracellular split tyrosine kinase domain.

In certain examples, the inhibitor of FGF comprises AZD4547. AZD4547 is a selective small molecule FGF inhibitor with potent antitumor activity. AZD4547 has the chemical structure as shown in Formula IV.

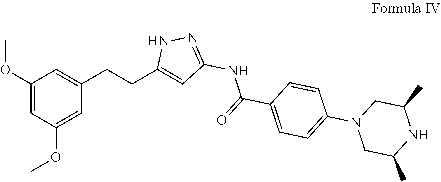 [SMILES: COc1cc(CCc2cc(NC(=O)c3ccc(N4C[C@@H](C)N[C@@H](C)C4)cc3)n[nH]2)cc(OC)c1]

In specific examples, the inhibitor of the CREB-heparan sulfate-FGF axis comprises SEQ ID NOS. 1-22. These SEQ ID NOS. correspond to sequences of shRNA that can be used to knockdown the genes that correspond to synthesis of heparan sulfate. These genes include, but are not limited to, EXT1, Ndst1, CREB1, hs2st1, hs3st1, and hs6st1.

In some examples, the inhibitor of the CREB-heparan sulfate-FGF axis comprises SEQ ID NOS. 1-4. These SEQ ID NOS. correspond to sequences of shRNA that can be used to knockdown EXT1. EXT1, also referred to as exostosin glycosyltransferase 1, is a gene that provides instructions for producing a protein called exostosin-1. This protein is found in a cell structure called the Golgi apparatus, which modifies newly produced enzymes and other proteins. In the Golgi apparatus, exostosin-1 binds to another protein, exostosin-2, to form a complex that modifies heparan sulfate.

In some examples, the inhibitor of the CREB-heparan sulfate-FGF axis comprises SEQ ID NO. 23. This SEQ ID NO. corresponds to a ras-related nuclear protein (RAN) sequence for knockout of EXT1.

In further examples, the inhibitor of the CREB-heparan sulfate-FGF axis comprises SEQ ID NOS. 24-25. These SEQ ID NOS. correspond to RAN sequences for knockout of Ndst1. Ndst 1, also referred to as N-deacetylase and N-sulfotransferase 1, is a gene that encodes a member of the heparan sulfate/heparin GlcNAc N-deacetylase/N-sulfotransferase family. The encoded enzyme is a type II transmembrane protein that resides in the Golgi apparatus. The encoded protein catalyzes the transfer of sulfate from 3′-phosphoadenosine 5′-phosphosulfate to nitrogen of glucosamine in heparan sulfate. Alternative splicing results in multiple transcript variants.

In further examples, the inhibitor of the CREB-heparan sulfate-FGF axis is administered in combination with cancer therapy.

In certain examples, the cancer therapy comprises surgery, chemotherapy, immunotherapy, ionizing radiation, or a combination thereof.

In specific examples, the surfers comprises stem cell transplantation (SCT). Stem cell transplantation (SCT), also referred to as a bone marrow transplant, is a procedure in which a subject receives healthy stem cells to replace damaged stem cells. This can include autologous transplantation in which the transplantation uses the subject's own stem cells. SCT can also include allogeneic transplantation, which uses stem cells from a donor.

In autologous transplantation, the subject stem cells are collected and stored. The cells are frozen and then returned to the subject after receiving intensive high-dose chemotherapy either with or without radiation therapy. This procedure can be used in clinically symptomatic subjects that are fit, young, and have few or no coexisting illnesses.

In some examples, chemotherapy can include R—CHOP (Rituxan, cyclophosphamide, doxorubicin, vincristine, and prednisone), VcR-CAP (bortezomib, rituximab, cyclophosphamide, doxorubicin, and prednisone), R-hyperCVAD (rituximab, cyclophosphamide, vincristine, doxorubicin, and dexamethasone alternating with high-dose cytarabine and methotrexate), B+R (bendamustine and rituximab), R-FCM (rituximab, fludarabine, cyclophosphamide, and mitoxantrone), R-DHAP (rituximab, dexamethasone, cytarabine and cisplatin), R—CVP (rituximab, cyclophosphamide, vincristine, and prednisone), or R—CBP (rituximab, cyclophosphamide, bortezomib, and prednisone), or any combination thereof. These chemotherapies can be administered intravenously or via mouth.

In further examples, cancer therapy can include R—CHOP followed by an autologous SCT, R—CHOP followed by higher doses of cytarabine and further followed by an autologous stem cell transplant, or R-hyperCVAD with autologous SCT, or any combination thereof.

In some examples, the method further comprises administering an additional therapeutic agent. In further examples, the additional therapeutic agent comprises abiraterone acetate, apalutamide, bicalutamide, cabazitaxel, darolutamide, degarelix, docetaxel, enzalutamide, flutamide, goserelin acetate, leuprolide acetate, lutetium Lu 177 vipivotide tetraxetan, mitoxantrone hydrochloride, nilutamide, olaparib, radium 223 dichloride, relugolix, rucaparib camsylate, sipuleucel-t, or any combination thereof. In some examples, the additional therapeutic agent is a form of chemotherapy.

In certain examples, the additional therapeutic agent comprises docetaxel. Docetaxel is a type of taxane, wherein taxanes interfere with structures in a cell called microtubules that help move chromosomes during mitosis. Docetaxel stops the growth of cancer cells and other dividing cells by blocking cell division. Docetaxel has been used as treatment for patients with castration-resistant prostate cancer for palliation and prolongation of life. Docetaxel can be administered via IV for a single dose on day 1 of a 21-day cycle for anywhere from six to ten cycles for example.

In specific examples, the Pten-loss induced cancer is a solid tumor. Solid tumors that are cancerous are mainly of monoclonal origin and manifest six essential alterations: growth signals, intensification of anti-growth signals, cell and tissue apoptosis or necrosis, limitless replicative and proliferative potential, prolonged angiogenesis, and organ/tissue invasion followed by metastasis. Solid tumors can include adenocarcinoma, small cell carcinoma, neuroendocrine tumor, transitional cell carcinoma, or sarcoma, as described below.

In some examples, the Pten-loss induced cancer comprises prostate cancer. Prostate cancer is cancer that occurs in the prostate. The prostate is a small walnut-shaped gland in males that produces the seminal fluid that nourishes and transports sperm. Prostate cancer develops when abnormal cells form and grow in the prostate gland. Prostate cancer can have no or few symptoms in early stages and in more advanced cases can cause signs and symptoms such as trouble urinating, decreased force in the stream of urine, blood in the urine, blood in the semen, bone pain, losing weight without trying, erectile dysfunction, or any combination thereof. Complications of prostate cancer can include, for example, metastasizing of the cancer, incontinence, erectile dysfunction, or any combination thereof. Prostate cancer can metastasize to nearby organs and tissues such as the bladder or rectum, as well as to other parts of the body. If an abnormal cancerous growth on the prostate is removed, it can still grow back, and if prostate cancer spreads far beyond the prostate, it can be life threatening.

In further examples, the prostate cancer comprises Stage I, Stage II, Stage III, or Stage IV prostate cancer. Stage I and Stage II prostate cancer refer to when the cancer has not spread beyond the prostate. This can also be referred to as “early-stage” or “localized” prostate cancer. Stage III prostate cancer has spread outside the prostate, but only to nearby tissues. This can be called “locally advanced prostate cancer”. Stage IV prostate cancer has spread outside the prostate to other parts such as the lymph nodes, bones, liver, or lungs. This stage can be called “advanced prostate cancer”.

In certain examples, the prostate cancer comprises adenocarcinoma, small cell carcinoma, neuroendocrine tumor, transitional cell carcinoma, sarcoma, or any combination thereof.

Adenocarcinomas of the prostate are the most common type of prostate cancer. They develop in the gland cells that line the prostate gland and the tubes of the prostate gland.

Small cell carcinomas of the prostate develop from neuroendocrine cells of the prostate. These cells do not make prostate specific antigen (PSA), an antigen that can be tested via blood test in prostate cancer screening, which can create difficulty when screening for prostate cancer. This is one of the more severe types of prostate cancer.

Neuroendocrine tumor prostate cancer is an aggressively histologic subtype of prostate cancer that most commonly arises in later stages of prostate cancer as a mechanism of treatment resistance.

Transitional cell carcinoma prostate cancer is a rare form of prostate cancer that starts in the section of the urethra that runs through the prostate gland or in the prostate ducts. This type of prostate cancer tends to be very aggressive and can spread easily to other parts of the prostate gland, the bladder, and the seminal vesicles, for example.

Sarcoma prostate cancer represents a relatively low number of primary prostate cancers. Prostate sarcomas are a heterogeneous group of tumors arising from mesenchymal cells in and around the prostate, which differs from prostate adenocarcinoma which derives from the glandular tissue.

TABLE 1
Sequences
shRNA sequences (knockout)

| | | | | |
|---|---|---|---|---|
| SEQ | 1 | EXT1- | Sense | 5′ CCGG AAGTCTCCACTTGTGGAACAA |
| ID | | shRNA | | CTCGAG |
| NO. | | #1 | | TTGTTCCACAAGTGGAGACTTTTTTTG 3′ |
| SEQ | 2 | EXT1- | Anti- | 5′ AATTCAAAAA |
| ID | | shRNA | sense | AAGTCTCCACTTGTGGAACAA CTCGAG |
| NO. | | #1 | | TTGTTCCACAAGTGGAGACTT 3′ |
| SEQ | 3 | EXT1- | Sense | 5′ CCGG AA GCAGACACAATTCTTGTGG |
| ID | | shRNA | | CTCGAG CCACAAGAATTGTGTCTGC |
| NO. | | #2 | | TTTTTTTG 3′ |
| SEQ | 4 | EXT1- | Anti- | 5′ AATTCAAAAA AA |
| ID | | shRNA | sense | GCAGACACAATTCTTGTGG CTCGAG |
| NO. | | #2 | | CCACAAGAATTGTGTCTGC TT 3′ |
| SEQ | 5 | PLKO- | Sense | 5′ CCGG CCTAAGGTTAAGTCGCCCTCG |
| ID | | scramble | | CTCGAG |
| NO. | | | | CGAGGGCGACTTAACCTTAGGTTTTTG 3′ |
| SEQ | 6 | PLKO- | Anti- | 5′ AATTCAAAAA |
| ID | | scramble | sense | CCTAAGGTTAAGTCGCCCTCG CTCGAG |
| NO. | | | | CGAGGGCGACTTAACCTTAGG 3′ |
| SEQ | 7 | Ndst1- | Sense | 5′ CCGG AACACAAAGACATCTGGTCCA |
| ID | | shRNA | | CTCGAG TGGACCAGATGTCTTTGTG |
| NO. | | #1 | | TTTTTTTG 3′ |
| SEQ | 8 | Ndst1- | Anti- | 5′ AATTCAAAAA |
| ID | | shRNA | sense | AACACAAAGACATCTGGTCCA CTCGAG |
| NO. | | #1 | | TGGACCAGATGTCTTTGTGTT 3′ |
| SEQ | 9 | Ndst1- | Sense | 5′ CCGG AACTGGTGGACATTGATGACA |
| ID | | shRNA | | CTCGAG TGTCATCAATGTCCACCAG |
| NO. | | #2 | | TTTTTTTG 3′ |
| SEQ | 10 | Ndst1- | Anti- | 5′ AATTCAAAAA |
| ID | | shRNA | sense | AACTGGTGGACATTGATGACA CTCGAG |
| NO. | | #2 | | TGTCATCAATGTCCACCAGTT 3′ |
| SEQ | 11 | Ndst1- | Sense | 5′ CCGG AAGGCGTTTGATCCAAAGAAA |
| ID | | shRNA | | CTCGAG TTTCTTTGGATCAAACGCC |
| NO. | | #3 | | TTTTTTTG 3′ |
| SEQ | 12 | Ndst1- | Anti- | 5′ AATTCAAAAA |
| ID | | shRNA | sense | AAGGCGTTTGATCCAAAGAAA CTCGAG |
| NO. | | #3 | | TTTCTTTGGATCAAACGCCTT 3′ |
| SEQ | 13 | CREB1 | Sense | 5′ CCGG AAGAGCAATACAGCTGGCTAA |
| ID | | shRNA | | CTCGAG TTAGCCAGCTGTATTGCTC |
| NO. | | #1 | | TTTTTTTG 3′ |
| SEQ | 14 | CREB1 | Anti- | 5′ AATTCAAAAA |
| ID | | shRNA | sense | AAGAGCAATACAGCTGGCTAA CTCGAG |
| NO. | | #1 | | TTAGCCAGCTGTATTGCTCTT 3′ |
| SEQ | 15 | CREB1 | Sense | 5′ CCGG AACCAAGTTGTTGTTCAAGCT |
| ID | | shRNA | | CTCGAG AGCTTGAACAACAACTTGG |
| NO. | | #2 | | TTTTTTTG 3′ |
| SEQ | 16 | CREB1 | Anti- | 5′ AATTCAAAAA |
| ID | | shRNA | sense | AACCAAGTTGTTGTTCAAGCT CTCGAG |
| NO. | | #2 | | AGCTTGAACAACAACTTGGTT 3′ |

| | | | |
|---|---|---|---|
| SEQ | 17 | hs2st1-shRNA-F | 5′CCGGAAGCCTATGACCTGTGTGCAACTCG |
| ID | | | AGTTGCACACAGGTCATAGGCTTTTTTTG 3′ |
| NO. | | | |
| SEQ | 18 | hs2st1-shRNA-R | 5′AATTCAAAAAAAGCCTATGACCTGTGTGC |
| ID | | | AACTCGAGTTGCACACAGGTCATAGGCTT 3′ |
| NO. | | | |
| SEQ | 19 | hs3st1-shRNA-F | 5′CCGGAA GCGTGCTATCTGACTACAC |
| ID | | | CTCGAG GTGTAGTCAGATAGCACGC |
| NO. | | | TTTTTTTG 3′ |
| SEQ | 20 | hs3st1-shRNA-R | 5′AATTCAAAAAAA |
| ID | | | GCGTGCTATCTGACTACAC CTCGAG |
| NO. | | | GTGTAGTCAGATAGCACGCTT 3′ |
| SEQ | 21 | hs6st1-shRNA-F | 5′ |
| ID | | | CCGGAACCAGGAAGTTCTACTACATCTCG |
| NO. | | | AGATGTAGTAGAACTTCCTGGTTTTTTTG 3′ |
| SEQ | 22 | hs6st1-shRNA-R | 5′ AATTCAAAAAAACCAGGAAGTTCTACTA |
| ID | | | CATCTCGAGATGTAGTAGAACTTCCTGGT |
| NO. | | | T 3′ |

RAN sequences for CRISPR-CAS9 (knockout)

| | | | |
|---|---|---|---|
| SEQ | 23 | sgEXT1 | GCAGACACAATTCTTGTGGGAGG |
| ID | | | |
| NO. | | | |
| SEQ | 24 | sgNdst1-F | 5′ CACCGCCGCTGCTCTACGTGACGCG 3′ |
| ID | | | |
| NO. | | | |
| SEQ | 25 | sgNdst1-R | 5′ AAACCGCGTCACGTAGAGCAGCGGC 3′ |
| ID | | | |
| NO. | | | |

CRISPR/gRNA-directed synergistic activation mediator (SAM)

| | | | |
|---|---|---|---|
| SEQ | 26 | sgSAM CREB- | 5′-CACCG AGCGGCCCCGCCCCCGCGCT-3′ |
| ID | | 24F | |
| NO. | | | |
| SEQ | 27 | sgSAM CREB- | 5′-AAAC AGCGCGGGGGCGGGGCCGCTC-3′ |
| ID | | 24R | |
| NO. | | | |
| SEQ | 28 | sgSAM CREB- | 5′-CACCG AGGCGGGCTGAGCCCGGCGC-3′ |
| ID | | 119F | |
| NO. | | | |
| SEQ | 29 | sgSAM CREB- | 5′-AAAC GCGCCGGGCTCAGCCCGCCTC-3′ |
| ID | | 119R | |
| NO. | | | |
| SEQ | 30 | sgSAM CREB- | 5′-CACCG CCGAGGCGCCGGCGCGGCGT-3′ |
| ID | | 159F | |
| NO. | | | |
| SEQ | 31 | sgSAM CREB- | 5′-AAAC ACGCCGCGCCGGCGCCTCGGC-3′ |
| ID | | 159R | |
| NO. | | | |
| SEQ | 32 | sgSAM CREB- | 5′-CACCG CAGCGCTGTCGTGTCGGGAA-3′ |
| ID | | 40F | |
| NO. | | | |
| SEQ | 33 | sgSAM CREB- | 5′-AAAC TTCCCGACACGACAGCGCTGC-3′ |
| ID | | 40R | |
| NO. | | | |

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Pten-Loss Upregulates CREB-Heparan Sulfate-FGF Signaling to Promote Prostate Tumorigenesis and Targeting Potential for Therapy

### Introduction

The systematic bioinformatic example described herein uncovered that loss-of-function mutation of Pten, a potent humor suppressor, correlated with upregulated heparan sulfate expression in human prostate cancer specimens. In mouse model and cell culture studies, it was discovered that Pten-loss upregulated CREB-heparan sulfate signaling to promote prostate tumorigenesis, and pharmacological inhibition of CREP or heparan sulfate both efficiently blocked cancer growth in vitro and in vivo, revealing the CREB-heparan sulfate acid as a target for cancer treatment.

This example found that CREB-heparan sulfate axis mediates Pten-loss-induced prostate cancer tumorigenesis and can be efficiently targeted. The data discussed herein suggest that for Pten-loss induced cancer, targeting CREB-heparan sulfate axis was efficient to treat the patients.

It was observed that knockout of Pten in prostate epithelial cells upregulated the HS gene expression. Functional studies further observed that overexpression of Ext1 is one of the HS genes that is upregulated by Pten-loss induced prostate epithelial cell colony formation in soft agar, supporting the aberrant HS expression-induced by Pten-loss, thereby functionally promoting prostate cancer initiation. In therapeutic exploration studies, knockdown of Ext1 or pharmacological inhibition of HS by surfen or CREB blocked PCa growth in a xenograft model in immune-competent syngeneic FVB/NJ mice. In conclusion, this study delineated that Pten-loss upregulated CREB-HS signaling to promote prostate tumorigenesis, and the signaling axis was targeted for PCa treatment.

### Materials and Methods

### Heparan Sulfate (HS), Biosynthesis and Remodeling

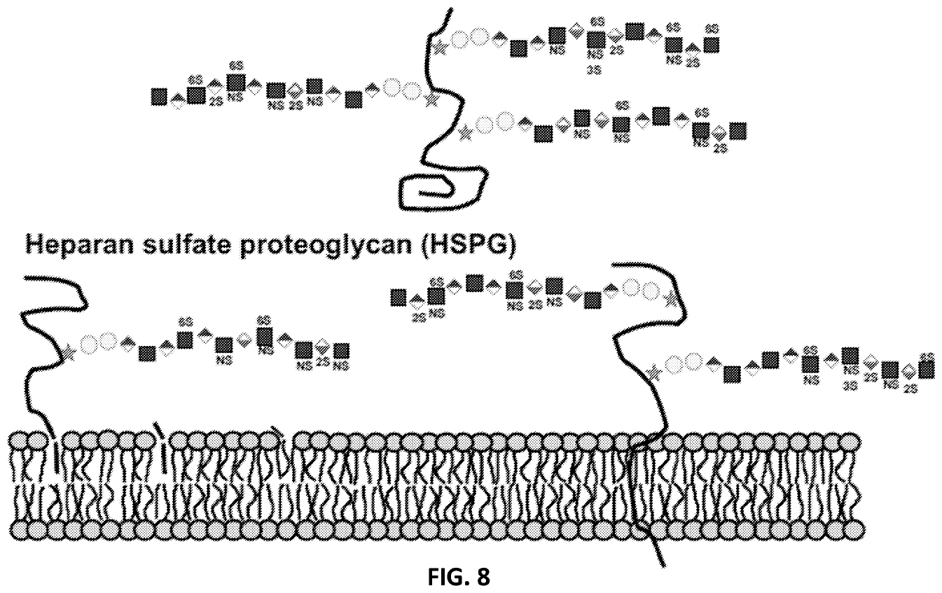

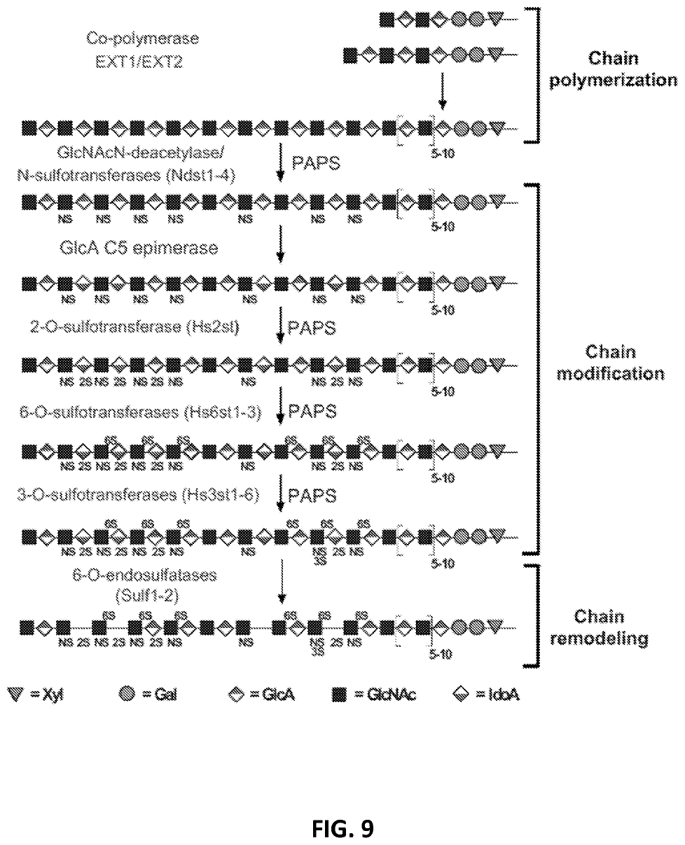

HS is a linear polysaccharide with multiple modifications which generate ligand-binding sites (fine structures). (FIG. 8) They are a key component of the cell microenvironment and play a role in cell-cell interactions, adhesion, migration, proliferation, and related signaling. HS was made via non-template driving biosynthesis and the final structure in the synthesis is determined by the expression profile of HS biosynthetic and remodeling genes/enzymes. (FIG. 9)

### Tumor Suppressor Pten-Loss in Prostate Cancer

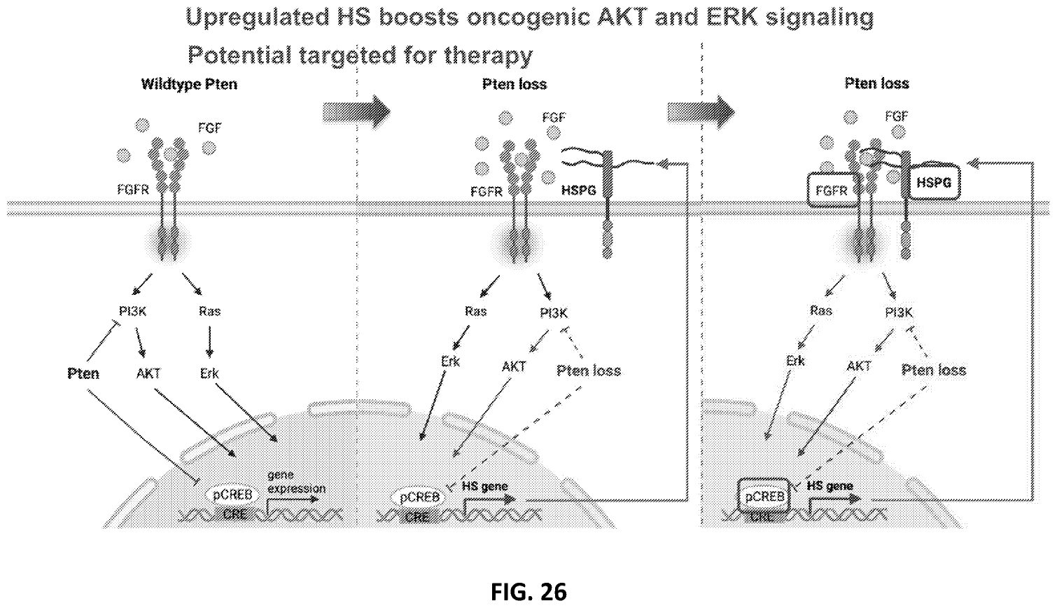

Genomic aberrations of the phosphatase and tensin homologue deleted on Chromosome 10 (PTEN) tumor suppressor gene are common in prostate cancer. Inactivation of PTEN by deletion or mutation was identified in this example in approximately 20% of primary prostate tumors and as many as 50% of castration-resistant tumors. Loss of PTEN function led to activation of the PI3K-AKT pathway and was strongly associated with oncological outcomes. (FIG. 26) Loss of PTEN increased transcription factor CREP phosphorylation to modulate gene expression. This function did not depend on PI3K/AKT pathway. The role of Pten-CREB pathway in cancer was not known.

### Causes of Abnormal HS Expression in Prostate Cancer

Pten-loss was associated with higher HS gene expression in prostate cancer patients and upregulates HS gene expression in a prostate cancer mouse model. Pten-loss upregulated HS gene expression in normal human prostate epithelial cells. Pten-loss acts through activation of CREB pathway, not AKT pathway, to upregulate HS gene expression in prostate epithelial cells. (FIG. 26)

### CONCLUSIONS

Herein, HS was utilized in Pten-loss to initiate and progress prostate cancer. HS overexpression induced prostate epithelial cell colony formation in soft agar and initiated prostate tumor formation in vivo, which suggested the Pten-loss-upregulated HS expression was an initiating factor for prostate cancer development. HS overexpression enhanced prostate tumor growth in vivo, suggesting that the Pten-loss-upregulated HS expression acted similarly to promote prostate tumorigenesis.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A method of treating a Pten-loss induced cancer in a subject in need thereof, the method comprising administering an inhibitor of a CREB-heparan sulfate-FGF axis.

2. The method of claim 1, wherein the inhibitor of the CREB-heparan sulfate-FGF axis comprises an inhibitor of CREB.

3. The method of claim 2, wherein the inhibitor of CREB comprises KG501.

4. The method of claim 2, wherein the inhibitor of CREB comprises 666-15.

5. The method of claim 1, wherein the inhibitor of the CREB-heparan sulfate-FGF axis comprises at least one inhibitor of heparan sulfate (HS) synthesis.

6. The method of claim 5, wherein the inhibitor of HS synthesis comprises bis-2-methyl-4-amino-quinolyl-6-carbamide.

7. The method of claim 1, wherein the inhibitor of the CREB-heparan sulfate-FGF axis comprises an inhibitor of fibroblast growth factor (FGF).

8. The method of claim 7, wherein the inhibitor of FGF comprises AZD4547.

9. The method of claim 1, wherein the inhibitor of the CREB-heparan sulfate-FGF axis comprises SEQ ID NOS. 1-22.

10. The method of claim 1, wherein the inhibitor of the CREB-heparan sulfate-FGF axis comprises SEQ ID NO. 23.

11. The method of claim 1, wherein the inhibitor of the CREB-heparan sulfate-FGF axis is administered in combination with a cancer therapy.

12. The method of claim 11, wherein the cancer therapy comprises surgery, chemotherapy, immunotherapy, ionizing radiation, or a combination thereof.

13. The method of claim 12, wherein the surgery comprises stem cell transplantation (SCT).

14. The method of claim 1, further comprising administering an additional therapeutic agent.

15. The method of claim 14, wherein the additional therapeutic agent comprises abiraterone acetate, apalutamide, bicalutamide, cabazitaxel, darolutamide, degarelix, docetaxel, enzalutamide, flutamide, goserelin acetate, leuprolide acetate, lutetium Lu 177 vipivotide tetraxetan, mitoxantrone hydrochloride, nilutamide, olaparib, radium 223 dichloride, relugolix, rucaparib camsylate, sipuleucel-t, or any combination thereof.

16. The method of claim 15, wherein the additional therapeutic agent comprises docetaxel.

17. The method of claim 1, wherein the Pten-loss induced cancer is a solid tumor.

18. The method of claim 1, wherein the Pten-loss induced cancer comprises prostate cancer.

19. The method of claim 18, wherein the prostate cancer comprises Stage I, Stage II, Stage III, or Stage IV prostate cancer.

20. The method of claim 18, wherein the prostate cancer comprises adenocarcinoma, small cell carcinoma, neuroendocrine tumor, transitional cell carcinoma, sarcoma, or any combination thereof.


SEQUENCE LISTING (33 sequences)

SEQ ID NO: 1 - RNA, 58 nt, synthetic construct
  source 1..58: mol_type = other RNA
ccggaagtctccacttgtggaacaactcgagttgttccacaagtggagactttttttg

SEQ ID NO: 2 - RNA, 58 nt, synthetic construct
  source 1..58: mol_type = other RNA
aattcaaaaaaagtctccacttgtggaacaactcgagttgttccacaagtggagactt

SEQ ID NO: 3 - RNA, 58 nt, synthetic construct
  source 1..58: mol_type = other RNA
ccggaagcagacacaattcttgtggctcgagccacaagaattgtgtctgctttttttg

SEQ ID NO: 4 - RNA, 58 nt, synthetic construct
  source 1..58: mol_type = other RNA
aattcaaaaaaagcagacacaattcttgtggctcgagccacaagaattgtgtctgctt

SEQ ID NO: 5 - RNA, 58 nt, synthetic construct
  source 1..58: mol_type = other RNA
ccggcctaaggttaagtcgccctcgctcgagcgagggcgacttaaccttaggtttttg

SEQ ID NO: 6 - RNA, 58 nt, synthetic construct
  source 1..58: mol_type = other RNA
aattcaaaaacctaaggttaagtcgccctcgctcgagcgagggcgacttaaccttagg

SEQ ID NO: 7 - RNA, 58 nt, synthetic construct
  source 1..58: mol_type = other RNA
ccggaacacaaagacatctggtccactcgagtggaccagatgtctttgtgtttttttg

SEQ ID NO: 8 - RNA, 58 nt, synthetic construct
  source 1..58: mol_type = other RNA
aattcaaaaaaacacaaagacatctggtccactcgagtggaccagatgtctttgtgtt

SEQ ID NO: 9 - RNA, 58 nt, synthetic construct
  source 1..58: mol_type = other RNA
ccggaactggtggacattgatgacactcgagtgtcatcaatgtccaccagtttttttg

SEQ ID NO: 10 - RNA, 58 nt, synthetic construct
  source 1..58: mol_type = other RNA
aattcaaaaaaactggtggacattgatgacactcgagtgtcatcaatgtccaccagtt

SEQ ID NO: 11 - RNA, 58 nt, synthetic construct
  source 1..58: mol_type = other RNA
ccggaaggcgtttgatccaaagaaactcgagtttctttggatcaaacgcctttttttg

SEQ ID NO: 12 - RNA, 58 nt, synthetic construct
  source 1..58: mol_type = other RNA
aattcaaaaaaaggcgtttgatccaaagaaactcgagtttctttggatcaaacgcctt

SEQ ID NO: 13 - RNA, 58 nt, synthetic construct
  source 1..58: mol_type = other RNA
ccggaagagcaatacagctggctaactcgagttagccagctgtattgctctttttttg

SEQ ID NO: 14 - RNA, 58 nt, synthetic construct
  source 1..58: mol_type = other RNA
aattcaaaaaaagagcaatacagctggctaactcgagttagccagctgtattgctctt

SEQ ID NO: 15 - RNA, 58 nt, synthetic construct
  source 1..58: mol_type = other RNA
ccggaaccaagttgttgttcaagctctcgagagcttgaacaacaacttggtttttttg

SEQ ID NO: 16 - RNA, 58 nt, synthetic construct
  source 1..58: mol_type = other RNA
aattcaaaaaaaccaagttgttgttcaagctctcgagagcttgaacaacaacttggtt

SEQ ID NO: 17 - RNA, 58 nt, synthetic construct
  source 1..58: mol_type = other RNA
ccggaagcctatgacctgtgtgcaactcgagttgcacacaggtcataggctttttttg

SEQ ID NO: 18 - RNA, 58 nt, synthetic construct
  source 1..58: mol_type = other RNA
aattcaaaaaaagcctatgacctgtgtgcaactcgagttgcacacaggtcataggctt

SEQ ID NO: 19 - RNA, 58 nt, synthetic construct
  source 1..58: mol_type = other RNA
ccggaagcgtgctatctgactacacctcgaggtgtagtcagatagcacgctttttttg

SEQ ID NO: 20 - RNA, 58 nt, synthetic construct
  source 1..58: mol_type = other RNA
aattcaaaaaaagcgtgctatctgactacacctcgaggtgtagtcagatagcacgctt

SEQ ID NO: 21 - RNA, 58 nt, synthetic construct
  source 1..58: mol_type = other RNA
ccggaaccaggaagttctactacatctcgagatgtagtagaacttcctggtttttttg

SEQ ID NO: 22 - RNA, 58 nt, synthetic construct
  source 1..58: mol_type = other RNA
aattcaaaaaaaccaggaagttctactacatctcgagatgtagtagaacttcctggtt

SEQ ID NO: 23 - RNA, 23 nt, synthetic construct
  source 1..23: mol_type = other RNA
gcagacacaattcttgtgggagg

SEQ ID NO: 24 - RNA, 25 nt, synthetic construct
  source 1..25: mol_type = other RNA
caccgccgctgctctacgtgacgcg

SEQ ID NO: 25 - RNA, 25 nt, synthetic construct
  source 1..25: mol_type = other RNA
aaaccgcgtcacgtagagcagcggc

SEQ ID NO: 26 - RNA, 25 nt, synthetic construct
  source 1..25: mol_type = other RNA
caccgagcggccccgcccccgcgct

SEQ ID NO: 27 - RNA, 25 nt, synthetic construct
  source 1..25: mol_type = other RNA
aaacagcgcgggggcggggccgctc

SEQ ID NO: 28 - RNA, 25 nt, synthetic construct
  source 1..25: mol_type = other RNA
caccgaggcgggctgagcccggcgc

SEQ ID NO: 29 - RNA, 25 nt, synthetic construct
  source 1..25: mol_type = other RNA
aaacgcgccgggctcagcccgcctc

SEQ ID NO: 30 - RNA, 25 nt, synthetic construct
  source 1..25: mol_type = other RNA
caccgccgaggcgccggcgcggcgt

SEQ ID NO: 31 - RNA, 25 nt, synthetic construct
  source 1..25: mol_type = other RNA
aaacacgccgcgccggcgcctcggc

SEQ ID NO: 32 - RNA, 25 nt, synthetic construct
  source 1..25: mol_type = other RNA
caccgcagcgctgtcgtgtcgggaa

SEQ ID NO: 33 - RNA, 25 nt, synthetic construct
  source 1..25: mol_type = other RNA
aaacttcccgacacgacagcgctgc